(19) United States
(12) Patent Application Publication
(10) Pub. No.: US 2026/0235274 A1
(43) Pub. Date: Aug. 13, 2026

(54) ULTRA-MICROLIGHT TRANSMISSION DEVICE HAVING DOUBLE HOUSING STRUCTURE

(71) Applicant: BIOLIGHT CORPORATION, Hanam-si, Gyeonggi-do (KR)

(72) Inventors: Mi Jung PARK, Seoul (KR); Sang-Ik YUN, Seoul (KR); Haelim JANG, Gunpo-si (KR); Jeong Su YANG, Seongnam-si (KR); Seo Hyun KIM, Seoul (KR)

(73) Assignee: BIOLIGHT CORPORATION, Hanam-si, Gyeonggi-do (KR)

(21) Appl. No.: 18/713,974

(22) PCT Filed: Nov. 28, 2022
(86) PCT No.: PCT/KR2022/018922

§ 371 Date: Apr. 13, 2026

(30) Foreign Application Priority Data
Nov. 26, 2021 (KR) 10-2021-0165847
Nov. 25, 2022 (KR) 10-2022-0160294

(51) Int. Cl. F21V 9/00; A01K 29/00; F21V 7/00; F21V 29/70

(52) U.S. Cl. CPC F21V 9/00; A01K 29/00; F21V 7/00; F21V 29/70

(57) ABSTRACT

Disclosed is an ultra micro light transmission device having a double housing structure according to various embodiments of the present invention to realize the above-mentioned objectives. The ultra micro light transmission device may comprise: a light source which generates light; a first housing including an inner space and performing spectroscopy and diffuse reflection on light introduced into the inner space; a second housing having a hollow interior and provided in the inner space of the first housing; a first filter unit which converts the spectral and diffusely reflected light; and a second filter unit which diffracts and interferes the converted light.

### TECHNICAL FIELD

The present invention relates to an ultra-microlight transmission device, and more specifically, to a device which generates and provides light that maximizes a cell proliferation effect.

### BACKGROUND ART

Ultra-microlight is light or energy that has polychromatic wavelengths in a visible light spectrum band and of which intensity is so weak to have brightness corresponding to 1/500,000 of brightness of a general fluorescent lamp. This ultra-microlight is at least 1,000 times weaker than bioluminescence and thus has excellent efficiency and safety. The possibility that the ultra-microlight could affect living creatures was first raised in academia in the 1930s, and thereafter, Popp, a German photobiologist, published experimental results showing that information exchange between cells occurred through ultra-microlight. Based on this background, as a result of many years of research performed by radiating ultra-microlight onto living creatures through an ultra-microlight generator, the safety and usefulness of the ultra-microlight was confirmed.

Ultra-microlight emitted from living creatures has very weak intensity and thus is referred to as ultra weak photon emission or biophoton emission. The phenomenon of biophoton emission is related to reactive oxygen species (ROS) generated during a normal metabolic process of living creatures. These ROS are formed as natural byproducts of oxygen from normal metabolism and play important roles in cell signaling and homeostasis.

For example, ultra-microlight can activate the biometabolism of living creatures and strengthen immunity. For a more specific example, ultra-microlight generated through an ultra-microlight generator may be radiated onto livestock, and the ultra-microlight may be absorbed into the body of the livestock to activate metabolism to increase cell proliferation and protein synthesis, which may improve immunity. That is, the ultra-microlight can improving the immunity and anti-aging and antioxidant abilities of living creatures to provide various effects of increasing body weight and shortening a market age. Korean Patent Publication No. 10-2019-0127223 discloses a method of strengthening immunity of shrimp through light radiation.

Meanwhile, in order for ultra-microlight to be radiated onto various living creatures and provided to maximize various effects, it is important that an ultra-microlight generator generates light in a more appropriate and efficient manner. For example, when photoelectron energization efficiency is increased, or thermionic (or photoelectric) emission is maximized, the efficiency of generating ultra-microlight can be further improved. That is, through the efficient structural features of a light generating device, ultra-microlight may be generated through improved energy efficiency or the fewer number of processing processes.

Therefore, in the art, there may be a demand for a light radiation device that generates light that is more excellent for providing bioenergy through optimal efficiency.

### DETAILED DESCRIPTION OF INVENTION

### Technical Problem

The present invention is directed to solving the above problems and providing an ultra-microlight transmission device which generates and provides ultra-microlight with improved cell proliferation efficiency.

The technical problems to be solved by the present invention are not limited to the above-described problems, and any other technical problems that are not described herein will be clearly understood from the following description by those skilled in the art to which the present invention pertains.

### Technical Solution

In order to solve the above problems, according to one embodiment of the present invention, there is provided an ultra-microlight transmission device having a double housing structure, including a light source configured to generate light, a first housing which includes an interior space and performs spectroscopy and diffuse reflection on light introduced into the interior space, a second housing which has a shape with a hollow interior and is provided in the interior space of the first housing, a first filter unit configured to convert the spectroscopic and diffusely reflected light into monochromatic light, and a second filter unit configured to cause diffraction and interference for the converted light.

In an alternative embodiment, at least a portion of the light generated by the light source may be directly emitted to the outside through the hollow interior of the second housing.

In an alternative embodiment, the ultra-microlight transmission device may further include a heat radiation member configured to absorb heat generated by the light source and transfer the absorbed heat to the interior space.

In an alternative embodiment, the first housing may include an outer wall prism provided therein and configured to perform spectroscopy and diffuse reflection on the introduced light in multiple directions, the second housing may include an inner wall prism provided at an outer circumference thereof and configured to perform spectroscopy and diffuse reflection on the introduced light in multiple directions, and the light spectroscopic and diffusely reflected by the outer wall prism and the inner wall prism may be radiated to the housing to emit photoelectrons to the interior space.

In an alternative embodiment, an inner wall of the first housing may be made of a stainless steel material, and the outer wall prism may be made of an acrylic material and supported on the inner wall.

In an alternative embodiment, the second housing may further include an internal electromagnetic wave generator provided in the hollow interior.

In an alternative embodiment, each of the first filter unit and the second filter unit may be provided to have an inner diameter that is greater than an outer diameter of the second housing and provided to pass through the second housing.

In an alternative embodiment, the second filter unit may adjust the converted light by causing continuous diffraction and interference through a plurality of prism discs.

In an alternative embodiment, the ultra-microlight transmission device may further include a third filter unit configured to perform filtering on light transmitted from the second filter unit, wherein the third filter unit is made of a black body acrylic plate material and filters light with predetermined energy intensity among the light transmitted from the second filter unit to emit the filtered light to the outside.

In an alternative embodiment, the ultra-microlight transmission device may further include one or more lenses configured to refract the light generated by the light source and transmit the refracted light to the interior space.

In an alternative embodiment, the ultra-microlight transmission device may further include a diffusion plate which is provided at a predetermined distance from the light source and diffuses the light generated by the light source to transmit the diffused light to the interior space.

In an alternative embodiment, the ultra-microlight transmission device may further include an electromagnetic wave generator provided to surround an outer surface of the first housing and configured to generate electromagnetic waves, and a blocking film provided to surround an outer surface of the electromagnetic wave generator and configured to block one-directional movement of the electromagnetic waves.

In an alternative embodiment, the ultra-microlight transmission device may further include a metal plate provided in one area of the interior space.

According to another embodiment of the present invention, there is provided a method of generating light energy, the method including radiating light generated by a light source to an interior space of a first housing, performing spectroscopy and diffuse reflection on the light introduced into the interior space of the first housing, performing conversion on the spectroscopic and diffusely reflected light through a first filter unit, and causing diffraction and interference for the converted light through a second filter unit, wherein a second housing having a hollow interior is provided in the interior space of the first housing.

Other specific details of the invention are included in the detailed description and drawings.

### Effects of Invention

According to various embodiments of the present invention, it is possible to provide an ultra-microlight transmission device that generates and provides ultra-microlight with improved cell proliferation efficiency.

The effects of the present invention are not limited to the effects described above, and other effects that are not described will be clearly understood from the following description by those skilled in the art.

### BRIEF DESCRIPTION OF DRAWINGS

Various aspects are now described with reference to the drawings, wherein like reference numerals are used to generally refer to similar components. In the following embodiments, for purposes of description, numerous specific detailed items are presented to provide a thorough understanding of one or more aspects. However, it may be apparent that such aspect(s) may be performed without these detailed items.

FIG. 1 is schematic view of a system for improving cell proliferation efficiency using an ultra-microlight transmission device according to one embodiment of the present invention.

FIG. 2 is an exemplary cross-sectional view of an ultra-microlight transmission device having a double housing structure according to one embodiment of the present invention.

FIG. 3 is an exemplary view illustrating a second housing according to one embodiment of the present invention.

FIG. 4 shows exemplary views for comparing energies emitted from a conventional ultra-microlight transmission device and the ultra-microlight transmission device of the present invention according to one embodiment of the present invention.

FIG. 5 is an exemplary cross-sectional view of an ultra-microlight transmission device including one or more lenses according to one embodiment of the present invention.

FIG. 6 is an exemplary cross-sectional view of an ultra-microlight transmission device including a diffusion plate according to one embodiment of the present invention.

FIG. 7 is an exemplary cross-sectional view of an ultra-microlight transmission device including an electromagnetic wave generator according to one embodiment of the present invention.

FIG. 8 is an exemplary cross-sectional view of an ultra-microlight transmission device including a metal plate according to one embodiment of the present invention.

FIG. 9 is a flowchart illustrating a method of generating light energy according to one embodiment of the present invention.

### BEST MODE FOR EMBODIMENT OF INVENTION

Various embodiments and/or aspects will be described with reference to the accompanying drawings below. In the following description, for purposes of the description, numerous specific detailed items are presented to help overall understanding of one or more aspects. However, it will also be appreciated by those skilled in the art that the aspect(s) may be carried out without these detailed items. The following disclosure and the accompanying drawings disclose specific exemplary aspects of one or more aspects in detail. However, these aspects are exemplary, some of the various methods in the principles of the various aspects may be used, and the disclosed descriptions are intended to include all these aspects and their equivalents. Specifically, as used herein, “embodiment,” “example,” “aspect,” “exemplary,” and the like are not to be understood as any described aspect or design being better or more advantageous than other aspects or designs.

Hereinafter, the same reference numerals denote the same or similar components regardless of the reference numerals, and overlapping descriptions thereof will be omitted. Further, in the description of the embodiments disclosed in the specification, when it is determined that detailed descriptions of related known technologies may obscure the principle of the embodiments disclosed in the specification, the detailed description thereof will be omitted. In addition, the accompanying drawings are only for easy understanding of the embodiments disclosed in the specification, and the technical spirit disclosed in the specification is not limited by the accompanying drawings.

Although first, second, and the like are used to describe various elements or components, these elements or components are not limited by these terms. These terms are only used to distinguish one element or component from another. Accordingly, a first element or component to be mentioned below may be a second element or component within the spirit of the present invention.

Unless otherwise defined, all terms (including technical and scientific terms) used herein have the same meaning as commonly understood by one having ordinary knowledge in the art to which the present invention belongs. Further, terms defined in a commonly used dictionary are not to be interpreted ideally or excessively unless otherwise defined.

In addition, the term “or” is intended to mean an inclusive “or” rather than an exclusive “or.” That is, unless otherwise specified or clear from context, “X uses A or B” is intended to mean one of the natural implicit substitutions. That is, when X uses A, X uses B, or X uses both A and B, “X uses A or B” may be applied to either of these cases. It should also be understood that the term “and/or” as used herein refers to and includes all possible combinations of one or more of the listed related items.

Further, it should be understood that the terms “comprises” and/or “comprising” mean that the feature and/or component is present, but does not exclude the presence or addition of one or more other features, components, and/or groups thereof. In addition, unless otherwise specified or when it is not clear from the context as referring to a singular form, the singular form in the specification and claims should generally be construed to mean “one or more.”

When a certain component is mentioned as being “connected” or “linked” to another component, it should be understood that the certain component may be directly connected or linked to another component, but still another component may be present therebetween. On the other hand, when it is mentioned that a certain component is “directly connected” or “directly linked” to another element, it should be understood that there is no other certain component therebetween.

The suffixes “module” and “part” for the components used in the following description are given or mixed in consideration of only the ease of writing the specification, and do not have a distinct meaning or role by themselves.

Reference to an element or layer “above” or “on” another element or layer includes all cases in which the element or layer directly on another element or layer as well as the element or layer on another element or layer with still another element or layer therebetween. On the other hand, a case in which an element is referred to as “directly on” or “immediately on” refers to a case in which another element or layer is not interposed therebetween.

Spatially relative terms “below,” “beneath,” “lower,” “above,” “upper,” and the like may be used to easily describe a component or a correlation with other components as shown in the drawings. Spatially relative terms should be understood as terms including different directions of an element during use or operation in addition to a direction shown in the drawings.

For example, when components shown in the drawing are reversed, a component described as “below” or “beneath” another component may be placed “above” the other component. Thus, the example term “below” may include both orientations of below and above. Components may also be oriented in other directions, and accordingly, spatially relative terms may be interpreted according to orientation.

Objects and effects of the present invention, and technical configurations for achieving them will be apparent with reference to the embodiments to be described below in detail with the accompanying drawings. In the description of the present invention, when it is determined that detailed descriptions of a well-known function or configuration may unnecessarily obscure the gist of the present invention, the detailed descriptions thereof will be omitted. Further, the terms to be described below are terms defined in consideration of functions in the present invention and thus may vary according to intentions or customs of users and operators.

However, the present invention is not limited to the embodiments to be disclosed below and may be implemented in various different forms. Only the present embodiments are provided so that the present invention is complete, and to completely convey the scope of the disclosure to those skilled in the art, and the present invention is only defined by the scope of the claims. Accordingly, the definition should be made based on the content throughout the specification.

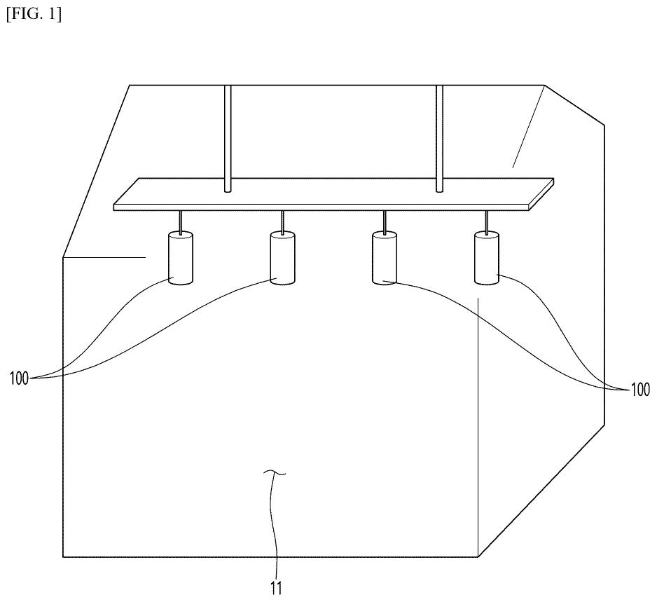

FIG. 1 is schematic view of a system for improving cell proliferation efficiency using an ultra-microlight transmission device according to one embodiment of the present invention.

As shown in FIG. 1, a plurality of ultra-microlight transmission devices 100 may be provided in one area of an indoor space 11. Here, the indoor space 11 may be a space in which living creatures are active. For example, the indoor space 11 may be a space in which living creatures related to livestock, such as cows, pigs, ducks, and chickens, are raised, but is not limited thereto.

The ultra-microlight transmission device 100 may be provided in one area of an upper side of the indoor space 11 and may radiate light for increasing the cell promotion efficiency of living creatures toward a lower portion at which the living creatures are active. The ultra-microlight transmission device 100 may be provided to have a certain separation distance from the living creatures. For example, the ultra-microlight transmission device 100 may be provided to have a separation distance of 1 m to 5 m from the living creatures in the indoor space. For a more specific example, the ultra-microlight transmission device 100 may be provided at a circle having a radius of 2 m from the living creatures to radiate light onto the living creatures. The description of specific numerical values of the position of the above-described transmission device is merely an example, and the present invention is not limited thereto.

The ultra-microlight transmission device 100 may generate ultra-microlight that contributes to an improvement in cell proliferation efficiency and may radiate the generated ultra-microlight onto living creatures. The ultra-microlight generated and radiated by the ultra-microlight transmission device 100 may be related to light that has a polychromatic wavelength in a visible light spectrum band and of which intensity is so weak to have brightness corresponding to 1/500,000 of brightness of a general fluorescent lamp.

The ultra-microlight with such a weak intensity can contribute to an improvement in cell proliferation efficiency of living creatures. For example, the ultra-microlight with such a weak intensity can activate the biometabolism of living creatures and strengthen immunity. For a more specific example, bioenergy light generated and emitted through the ultra-microlight transmission device may be radiated onto living creatures, and the bioenergy light may be absorbed into the body of the living creatures to activate metabolism to increase cell proliferation and protein synthesis, which may improve immunity. That is, bioenergy light can improve the immunity and anti-aging and antioxidant abilities of living creatures to provide various effects of increasing body weight and shortening a market age.

The ultra-microlight transmission device 100 of the present invention may generate ultra-microlight that is radiated onto living creatures to maximize various provided effects. To this end, the ultra-microlight transmission device 100 may be provided to have structural features for generating ultra-microlight through optimal efficiency. Optimal ultra-microlight may be ultra-microlight that maximizes the cell proliferation efficiency of living creatures or ultra-microlight generated with optimal efficiency.

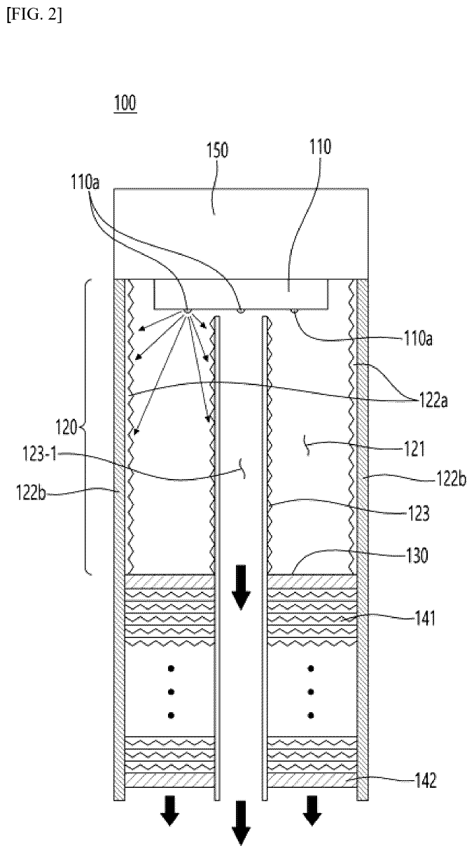

For example, in order to generate or emit optimal ultra-microlight, it may be important to maximize a photoelectric effect or thermionic emission. For a more specific example, when a photoelectric effect is maximized by increasing photoelectric emission efficiency in which photons are converted into photoelectrons, or thermionic emission efficiency is improved, ultra-microlight may be generated with less energy consumption. In other words, as photoelectric or thermionic emission efficiency is increased, the efficiency of generating ultra-microlight can be maximized. That is, the ultra-microlight transmission device 100 of the present invention may be implemented through a structure that maximizes a photoelectric effect and heat emission efficiency and thus may generate ultra-microlight through optimal efficiency such as minimizing energy consumption, thereby radiating the ultra-microlight onto living creatures. The structural features, configuration, action, and effects generated therethrough of an ultra-microlight transmission device that generates optimal ultra-microlight will be described in more detail below with reference to FIGS. 2 to 9.

FIG. 2 is an exemplary cross-sectional view of an ultra-microlight transmission device having a double housing structure according to one embodiment of the present invention. As shown in FIG. 2, an ultra-microlight transmission device 100 may include a light source 110, a first housing 120, a second housing 123, a first filter unit 130, a second filter unit 141, a third filter unit 142, and a heat radiation member 150. The above-described components are exemplary, and the scope of the present invention is not limited to the above-described components. That is, according to the implementation aspect of the embodiments of the present invention, additional components may be included, or some of the above-described components may be omitted.

According to one embodiment of the present invention, the ultra-microlight transmission device 100 may include the light source 110. The light source 110 may refer to a radiating body that emits infrared light, visible light, ultraviolet light, or the like. The light source 110 may directly or indirectly convert heat or electrical energy into radiant energy. For example, the light source 110 may generate light related to infrared light, visible light, ultraviolet light, and the like through light-emitting through combustion, light-emitting through electrical discharge, or light-emitting through a semiconductor. The specific description of a light generation method described above is merely an example, and the present invention is not limited thereto. The light source 110 may be disposed in one direction of the first housing 120 to transmit light to an interior space 121 of the first housing 120. In one embodiment, the light source 110 may be constituted through a plurality of light-emitting diode (LED) elements 110a. Each of the plurality of LED elements 110a may be a semiconductor element that generates and provides light through current.

According to one embodiment of the present invention, the ultra-microlight transmission device 100 may include the heat radiation member 150. A power source that applies power to the light source 110 may be provided in one area inside the heat radiation member 150. The heat radiation member 150 may diffuse heat generated by the power source. That is, the heat radiation member 150 may effectively control an increase in amount of heat generated inside electronic devices during a continuous use process, that is, a heat generation phenomenon.

The heat radiation member 150 may be made of a material with excellent thermal conductivity. As thermal conductivity becomes higher, heat energy may be well transmitted (that is, diffused) to other places, thereby effectively controlling generated heat. For example, the heat radiation member 150 may be made of a metal or ceramic material with high thermal conductivity. In addition, for example, the heat radiation member 150 may be made of a polymer composite material formed in such a manner that one of a carbon-based filler and a ceramic-based filler, which have excellent thermal conductivity, is used or a mixture thereof is uniformly spectroscopic in a polymer matrix to densely fill the polymer matrix, wherein the carbon-based filler includes graphite, carbon fiber, carbon nanotubes, or graphene, and the ceramic-based filler includes boron nitride, aluminum nitride, or alumina. The specific description of the materials constituting the above-described heat radiation member is merely an example, and the present invention is not limited thereto. According to an additional embodiment, the heat radiation member 150 is made of a material of which a thermal expansion coefficient is a predetermined level or less, thereby reducing the possibility of failure due to component defects due to heat generation.

The heat radiation member 150 may be positioned in one direction (for example, an upward direction) of the light source 110 and provided adjacent to the first housing 120. As shown in FIG. 2, the heat radiation member 150 may be provided in contact with one surface of the first housing 120, and thus heat generated in a process in which the light source 110 generates light may be transferred to the first housing 120. That is, the heat radiation member 150 may diffuse the generated heat into the first housing 120. In this case, the first housing 120 may form the interior space 121 in which a photoelectric effect or heat radiation operation occurs. As the heat radiation member 150 diffuses heat into the first housing 120, thermionic emission efficiency can be improved in the interior space 121 of the first housing 120, which may ultimately maximizes thermionic emission to maximize the efficiency of generating ultra-microlight.

According to one embodiment of the present invention, the ultra-microlight transmission device 100 may include the first housing 120. In the interior space 121 of the first housing 120, introduced light may be spectroscopic and diffusely reflected in multiple directions. As shown in FIG. 2, an outer wall prism 122a may be formed in the interior space 121 of the first housing 120 in an inward direction of the first housing 120, and light may be spectroscopic and diffusely reflected through the outer wall prism 122a to emit photoelectrons to the interior space 121.

Specifically, light generated by the light source 110 may be radiated to the interior space 121 of the first housing 120, and as the light strikes a wall in the interior space 121, photoelectrons may be generated. In this case, since the light itself emitted from the light source 110 is composed of photons with a variety of energy, an energy level of photoelectrons generated in the interior space 121 may also vary.

Specifically, light introduced from the light source 110 may be spectroscopic and diffusely reflected through the outer wall prism 122a of the first housing 120 to emit photoelectrons. Specifically, the outer wall prism 122a may be made of an acrylic material and formed in a shape of a figure on a plane that is not parallel to a side surface of the first housing 120. That is, the outer wall prism 122a may include a plurality of polygonal prisms protruding inward from a side wall of the first housing 120 in a shape in which at least one pair of faces are not parallel. For example, the plurality of polygonal prisms may have a triangular prism shape. However, the shape of the plurality of polygonal prisms constituting the wall prism is not limited thereto and may be implemented through various shapes such as a polygonal prism shape, a polygonal pyramid shape, a cone shape, and a spherical shape.

The plurality of polygonal prisms constituting the outer wall prism 122a may have various sizes ranging from several nanometers to several millimeters. When light radiated from the light source 110 is incident on the outer wall prism 122a (that is, each of the plurality of polygonal prisms), a degree of refraction varies according to a wavelength or frequency, which may cause dispersion. In other words, light is dispersed for each wavelength (for example, each energy level) through the outer wall prism 122a.

In addition, the first housing 120 may include an inner wall 122b that supports the outer wall prism 122a and is made of a metal material. According to one embodiment, the inner wall 122b may be made of a stainless steel material. As shown in FIG. 2, the inner wall 122b may be formed adjacent to an inner surface of the first housing 120 having a cylindrical shape, and the outer wall prism 122a constituted through the plurality of polygonal prisms may be formed using the inner wall 122b as a support. Accordingly, when light generated by the light source 110 is radiated to the first housing 120, the light passes through the outer wall prism 122a to be transmitted to the inner wall 122b.

The inner wall 122b may be made of a metal material and thus may bind electrons. Specifically, within the inner wall 122b, electrons may be bound (or confined) by a positive charge of an atomic nucleus and an electric force. Electrons bound to the inner wall 122b may be emitted by light with various wavelengths. That is, as light is transmitted, photoelectrons may be emitted. In this case, light transmitted to the inner wall 122b may be light dispersed into photons with a variety of energy through the outer wall prism 122a, and thus photoelectric emission can be maximized. That is, the photon absorption efficiency of the inner wall 122b may be increased through the outer wall prism 122a, and thus photoelectric emission can be maximized. In this case, since the light itself emitted from the light source 110 is composed of photons with a variety of energy, an energy level of photoelectrons generated in the interior space 121 may also vary.

According to an additional embodiment, the inner wall 122b may be made of an aluminum (Al) material. When the inner wall 122b is made of an aluminum material, photoelectronic emission efficiency can be further improved. Specifically, metals have their own intrinsic work function W and a limit frequency (or a threshold frequency). Here, the work function and the limit frequency may respectively mean the minimum energy and frequency of light that causes electrons bound to metal to be emitted. Aluminum has a work function of 4.06 eV to 4.26 eV, which may be lower than those of other metals. That is, when the inner wall 122b is made of an aluminum material, since the inner wall 122b has a low work function, the minimum energy of light for emitting photoelectrons may be reduced, and thus photoelectrons may be emitted through less light energy.

In addition, according to embodiments, a work function may also be important in thermionic emission. Thermionic emission may mean that charge carriers flow from the surface over a potential energy barrier by heat. Unlike a photoelectric effect, in thermionic emission, electrons may be emitted using heat instead of photons. Specifically, according to Richardson's law, the following equation holds:

$$J = {AT}^{2} {e}^{\frac{- W}{kT}}$$

Here, J may be a current density, T may be an absolute temperature, W may be a work function, K may be a Boltzmann constant, and A may be a Richardson constant. In other words, as a work function, which is energy that binds electrons, becomes lower, thermionic emission efficiency can be improved. Since aluminum has a work function of 4.06 eV to 4.26 eV, which is lower than those of other metals, heat energy required to emit thermoelectrons may be minimized, which makes it possible to emit theremoelectrons through relatively less heat energy.

In other words, when the inner wall 122b is made of an aluminum material, photoelectronic emission and thermoionic emission efficiencies can be improved. Improvements in photoelectronic emission and thermoionic emission efficiencies may ultimately contribute to an improvement in efficiency of generating ultra-microlight.

According to one embodiment of the present invention, the ultra-microlight transmission device 100 may include the second housing 123. The second housing 123 may have a shape with a hollow interior and may be provided in the interior space 121 of the first housing 120. For example, the second housing 123 may have a cylindrical shape with a hollow interior. Specifically, as shown in FIG. 2, the second housing 123 may be provided in the interior space 121 of the first housing 120. The second housing 123 may maximize photoelectronic emission and thermionic emission in the interior space 121, thereby maximizing the efficiency of generating ultra-microlight and simultaneously emitting light generated by the light source directly to the outside.

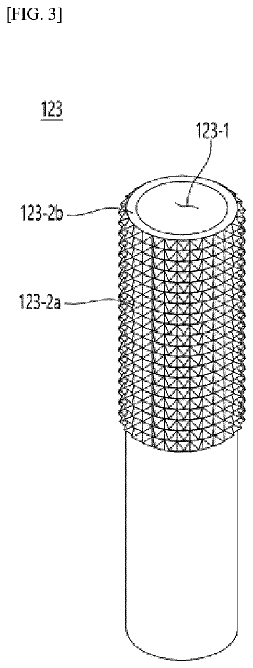

Specifically, as shown in FIG. 3, the second housing 123 may include a hollow interior 123-1 and an inner wall prism 123-2a formed along an outer circumference. Light emitted from the light source 110 may be transmitted to each of the hollow interior 123-1 and the interior space 121 formed between the first housing 120 and the second housing 123. In this case, when the light generated by the light source 110 moves into the hollow interior 123-1 of the second housing 123, the light is directly emitted to the outside. That is, the light generated by the light source 110 may be emitted to the outside through the hollow interior 123-1 of the second housing 123 without separate conversion, adjustment, and filtering processes.

In addition, the second housing 123 may include the inner wall prism 123-2a formed along the outer circumference. The inner wall prism 123-2a may be made of an acrylic material and provided in a shape of a figure on a plane that is not parallel to a side surface of the second housing 123. That is, the inner wall prism 123-2a may include a plurality of polygonal prisms protruding outward from an outer wall of the second housing 123 in a shape in which at least one pair of faces are not parallel. For example, the plurality of polygonal prisms may be provided in a triangular prism shape. However, the shape of the plurality of polygonal prisms constituting the inner wall prism is not limited thereto and may be implemented through various shapes such as a polygonal prism shape, a polygonal pyramid shape, a cone shape, and a spherical shape.

The plurality of polygonal prisms constituting the inner wall prism 123-2a may have various sizes ranging from several nanometers to several millimeters. When light radiated from the light source 110 is incident on the inner wall prism 123-2a (that is, each of the plurality of polygonal prisms), a degree of refraction varies according to a wavelength or frequency, which may cause dispersion. In other words, light is dispersed for each wavelength (for example, each energy level) through the inner wall prism 123-2a.

In addition, the second housing 123 may include an outer wall 123-2b that supports the inner wall prism 123-2a and is made of a metal material. According to one embodiment, the outer wall 123-2b may be made of a stainless steel material. As shown in FIG. 2, the outer wall 123-2b may be formed to correspond to an outer surface of the second housing 123 having a cylindrical shape, and the inner wall prism 123-2a constituted through the plurality of polygonal prisms may be formed using the outer wall 123-2b as a support. Accordingly, when light generated by the light source 110 is radiated to the interior space 121, the light passes through the inner wall prism 123-2a to be transmitted to the outer wall 123-2b.

The outer wall 123-2b may be made of a metal material and thus may bind electrons. Specifically, within the outer wall 123-2b, electrons may be bound (or confined) by a positive charge of an atomic nucleus and an electric force. Electrons bound to the outer wall 123-2b may be emitted by light with various wavelengths. That is, as light is transmitted, photoelectrons may be emitted. In this case, light transmitted to the outer wall 123-2b may be light dispersed into photons with a variety of energy through the inner wall prism 123-2a, and thus photoelectric emission can be maximized. That is, the photon absorption efficiency of the outer wall 123-2b may be increased through the inner wall prism 123-2a, and thus photoelectric emission can be maximized. In this case, since the light itself emitted from the light source 110 is composed of photons with a variety of energy, an energy level of photoelectrons generated in the interior space 121 may also vary.

That is, the ultra-microlight transmission device 100 of the present invention may be constituted through a double housing structure including the second housing 123 inside the first housing 120. In this case, an outer diameter of the second housing 123 may be less than an inner diameter of the first housing 120, and thus a space (that is, the interior space 121) may be formed between the first housing 120 and the second housing 123. In this case, the first housing 120 may be provided to include the outer wall prism 122a in an inward direction, and the second housing 123 may be provided to include the inner wall prism 123-2a in an outward direction. In other words, wall prisms formed between respective housings may be provided to face each other.

Accordingly, photoelectronic emission and thermionic emission efficiencies can be further maximized. Specifically, since an area of each wall prism (that is, each of the inner wall 122b and the outer wall 123-2b) made of a metal material that binds electrons is further increased, photoelectronic emission and thermal emission can be further maximized. In addition, since light transmitted to each wall prism made of a metal material is light dispersed into photons with a variety of energy through the outer wall prism 122a and the inner wall prism 123-2a, photoelectronic emission can be further maximized.

In summary, the wall prisms may be provided in the first housing 120 and the second housing 123 to face each other, and as a result, the efficiency of spectroscopic and diffusely reflected light can be increased and photoelectric and thermionic emission areas can be increased so that a photoelectric effect and a thermionic emission effect can be maximized.

According to one embodiment of the present invention, the ultra-microlight transmission device 100 may include the first filter unit 130. The first filter unit 130 may uniformly convert the spectroscopic and diffusely reflected light into monochromatic light to transmit the monochromatic light to the second filter unit 141.

More specifically, the first filter unit 130 may be made of an acrylic material. The first filter unit 130 is provided to have an inner diameter that is greater than the outer diameter of the second housing 123 and may be provided to pass through the second housing 123. For example, the first filter unit 130 may have an outer diameter corresponding to the inner diameter of the first housing 120, may have a thickness of 1 mm to 5 mm, and may pass through the second housing 123. That is, as shown in FIG. 2, the first filter unit 130 may be provided in a donut shape of which an inner diameter corresponds to the outer diameter of the second housing 123.

The first filter unit 130 may be provided to be connected to one end of the first housing 120 and may receive light from the interior space 121 between the first housing 120 and the second housing 123. The light received from the interior space 121 may be light spectroscopic and diffusely reflected through the outer wall prism 122a of the first housing 120 and the inner wall prism 123-2a of the second housing 123 (that is, light through which photoelectronic emission or thermionic emission is performed. Spectroscopic and diffusely reflected light has different white light characteristics according to the intensity and wavelength characteristics of light and thus may exhibit non-uniform color distribution characteristics. Accordingly, the first filter unit 130 may convert the spectroscopic and diffusely reflected light into uniform monochromatic light. For example, the first filter unit 130 may convert the spectroscopic and diffusely reflected light (that is, photoelectrons) into monochromatic light such as blue frequency energy. The first filter unit 130 may function as a color correction filter for light.

That is, light spectroscopic and diffusely reflected from the outer wall prism 122a of the first housing 120 and the inner wall prism 123-2a of the second housing 123 may be converted into uniform monochromatic light while passing through the first filter unit 130 and transmitted to the second filter unit 141 positioned in one direction (for example, downward direction in FIG. 2) of the first filter unit 130. Through the role of the color correction filter of the first filter unit 130, light with various characteristics may be converted into uniform light with the same characteristics.

According to one embodiment of the present invention, the ultra-microlight transmission device 100 may include the second filter unit 141. The second filter unit 141 may be provided to have an inner diameter that is greater than the outer diameter of the second housing 123 and provided to pass through the second housing 123. That is, as shown in FIG. 2, the second filter unit 141 may be provided in a donut shape of which an inner diameter corresponds to the outer diameter of the second housing 123.

According to one embodiment of the present invention, the ultra-microlight transmission device 100 may include the second filter unit 141 provided by stacking a plurality of prism discs. In addition, the ultra-microlight transmission device 100 may include the third filter unit 142 that filters light transmitted from the second filter unit 141.

In one embodiment, the second filter unit 141 may adjust light converted through continuous diffraction and interference through the plurality of prism discs (that is, light passing through the first filter unit). Specifically, as shown in FIG. 2, the second filter unit 141 may be implemented by stacking the plurality of prism discs.

The second filter unit 141 may be provided in contact with a side of the first filter unit 130 in one direction (for example, a downward direction) and provided in a form in which the plurality of prism discs are stacked. Converted light passing through the first filter unit 130 may be subjected to continuous diffraction and interference while passing through each layer of the second filter unit 141 and thus may be adjusted. Adjusting the converted light may mean that light is adjusted to have an optimal wavelength range, for example, so as to improve cell proliferation efficiency in living creatures. For a specific example, light may be adjusted by passing through the second filter unit 141 and thus may have a wavelength of 300 nm to 870 nm. Here, light with a wavelength of 300 nm to 870 nm may be appropriate light for increasing cell proliferation efficiency (for example, improving reproductive potential) of living creatures. According to one embodiment, the second filter unit 141 may adjust light to have various wavelength bands according to an aspect of the provision of the plurality of prism discs. That is, while passing through each layer (that is, the plurality of prism discs), through continuous diffraction and interference, light passing through the second filter unit 141 may be adjusted to have an appropriate wavelength to provide cell proliferation efficiency to living creatures.

In one embodiment, the third filter unit 142 may be made of a black body acrylic material. The black body acrylic material may function as a filter that transmits only light with a specific range of intensity. That is, the third filter unit 142 may allow only light with a certain range of intensity to be emitted to the outside through the black body acrylic material.

Specifically, the third filter unit 142 may emit ultra-microlight to the outside by filtering light with preset intensity among light transmitted from the second filter unit 141. Here, the preset intensity may refer to a range of light related to the optimal intensity for improving cell proliferation efficiency of living creatures. For example, light (that is, ultra-microlight) emitted through the third filter unit 142 may have an intensity of 10⁻¹⁸ W/cm² to 10⁻¹⁵ W/cm². In other words, light with an intensity of 10⁻¹⁸ W/cm² to 10⁻¹⁵ W/cm² may be light with optimal intensity for increasing cell proliferation efficiency of living creatures. For example, when light outside a range of 10⁻¹⁸ W/cm² to 10⁻¹⁵ W/cm² (for example, light with an intensity of 10⁻¹¹ W/cm²) is radiated onto living creatures, the light may not be appropriate light (that is, ultra-microlight) that increases cell proliferation efficiency of living creatures.

That is, the third filter unit 142 may filter light (for example, light in a specific wavelength band) passing through the second filter unit 141 such that only light with specific intensity is emitted to the outside. Accordingly, the light emitted to the outside may be ultra-microlight which is light with optimal intensity for increasing cell proliferation efficiency of living creatures.

According to one embodiment, light generated by the light source 110 may sequentially pass through the interior space 121, the first filter unit 130, the second filter unit 141, and the third filter unit 142 to be emitted to the outside. In summary, the heat radiation member 150 transfers (or diffuses) heat, which is generated in a process in which the light source 110 generates light, to the interior space 121 of the first housing 120, thereby maximizing thermionic emission efficiency in the interior space 121. In addition, the light emitted from the light source 110 is spectroscopic and diffusely reflected through the outer wall prism 122a and the inner wall prism 123-2a, thereby maximizing photoelectronic emission efficiency. Light related to photoelectronic and thermionic emission passes through the first filter unit 130, and in such a process, spectroscopic and diffusely reflected light may be uniformly converted into monochromatic light. While passing through the second filter unit 141 provided to include the plurality of prism discs, through continuous diffraction and interference, light converted into uniform monochromatic light after passing through the first filter unit 130 may be adjusted to have a specific wavelength range to be transmitted to the third filter unit 142. The third filter unit 142 may allow only light (that is, ultra-microlight), which has a certain level or more of energy intensity among light transmitted from the second filter unit 141, to be radiated to the outside of the ultra-microlight transmission device 100.

That is, ultra-microlight that increases cell proliferation efficiency in living creatures may be generated and emitted to the outside. Here, the ultra-microlight may be light converted and adjusted to have an optimal wavelength and intensity range for increasing cell proliferation efficiency of living creatures while passing through the second filter unit 141 and the third filter unit 142.

In addition, in a process of generating ultra-microlight, the heat radiation member 150 may transfer heat to the interior space 121 to maximize thermionic emission efficiency, thereby improving the efficiency of generating ultra-microlight. In addition, in a process of generating bioenergy light, photoelectronic emission efficiency is maximized through the outer wall prism 122a, thereby improving the efficiency of generating ultra-microlight. Additionally, since the outer wall prism 122a and the inner wall prism 123-2a are respectively included in the first housing 120 and the second housing 123, photoelectric and thermionic emission areas are increased, thereby maximizing photoelectronic emission and thermionic emission efficiencies.

That is, the ultra-microlight transmission device 100 of the present invention can generate ultra-microlight with optimal efficiency through structural features that maximize photoelectronic emission and thermionic emission.

According to one embodiment, the ultra-microlight transmission device 100 may emit LED light as well as ultra-microlight, thereby improving light emission efficiency. For example, in addition to ultra-microlight, additional light (for example, LED light) may be required for detecting a surrounding terrain or managing and supervising living creatures.

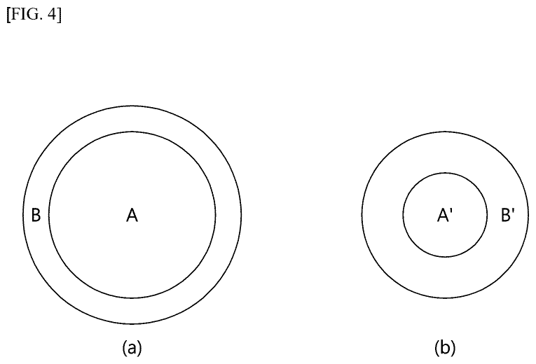

More specifically, in the case of a conventional or general ultra-microlight transmission device, separate light cannot be emitted other than ultra-weak light emission related to ultra-microlight. Accordingly, in the case of the conventional or general ultra-microlight transmission device, as shown in (a) of FIG. 4, an additional light source module may be provided around an outer circumferential surface of area A, through which ultra-microlight is emitted, to emit LED light. That is, as shown in (a) of FIG. 4, ultra-microlight related to ultra-microlight may be emitted through area A, and light may be emitted to correspond to area B through a separate light source module (or an LED module) provided on the outer circumferential surface of area A. In other words, since an additional light source module should be provided in addition to a light source used to generate ultra-microlight, energy consumption may be high, which may not be efficient.

On the other hand, the ultra-microlight transmission device 100 of the present invention can directly emit light generated by the light source 110 to the outside through the second housing 123 including the hollow interior 123-1. When light generated by the light source 110 moves into the hollow interior 123-1 of the second housing 123, the light is directly emitted to the outside. That is, the light generated by the light source 110 may be emitted to the outside without separate conversion, adjustment, and filtering processes. Specifically, as shown in (b) of FIG. 4, light (for example, LED light) generated by the light source 110 may be emitted to correspond to area A′, and at the same time, ultra-microlight generated by sequentially passing through the wall prism, the first filter unit 130, the second filter unit 141, and the third filter unit 142 may be emitted to correspond to area B′. In other words, both ultra-microlight and LED light may be generated and provided through one light source module, which may be more efficient. Therefore, the efficiency of generating ultra-microlight is maximized through the double housing structure as described above, and at the same time, LED light generation is possible without a separate light source module, thereby miniaturizing equipment and improving energy consumption efficiency.

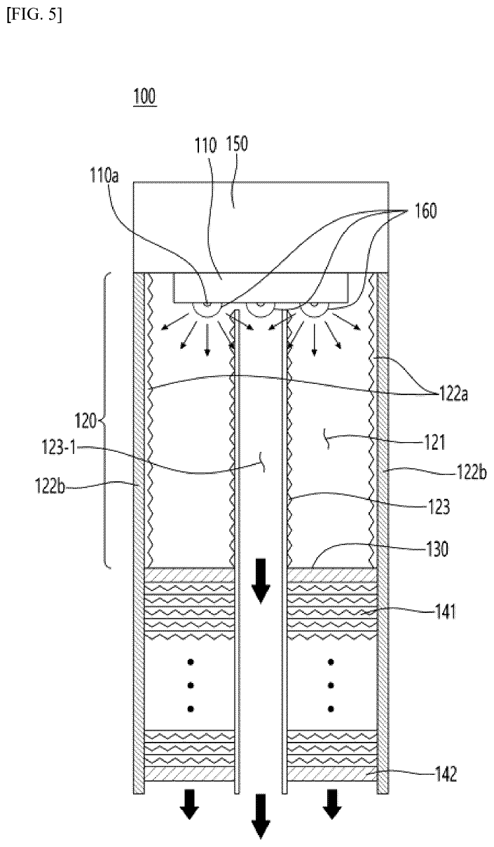

According to one embodiment of the present invention, the ultra-microlight transmission device 100 may include one or more lenses 160. The one or more lenses 160 will be described in more detail below with reference to FIG. 5.

As shown in FIG. 5, the one or more lenses 160 may be provided in contact with the light source 110 and may refract light generated by the light source 110 to transmit the refracted light to the interior space 121 between the first housing 120 and the second housing 123. Specifically, the light source 110 may be provided to include a plurality of LED elements 110a, each of which generates light. The one or more lenses 160 may each be provided to correspond to one of the plurality of LED elements 110a. The one or more lenses 160 may be provided in hemispherical shapes surrounding the plurality of LED elements. Each of the one or more lenses 160 may change an angle of light emitted from each of the plurality of LED elements.

According to one embodiment, each of the one or more lenses 160 may refract light emitted from each corresponding LED element to diffuse the refracted light in the interior space 121. In this case, light emitted from each LED element may pass through each lens to diffuse in multiple directions in the interior space 121. Accordingly, light transmitted to each of the outer wall prism 122a and the inner wall prism 123-2a can be maximized. As the light transmitted to each wall prism is improved, photoelectronic emission efficiency through each wall prism can be improved. That is, the one or more lenses 160 can maximize a photoelectric effect by diffusing and supplying light to the interior space 121 in which the wall prism is positioned (that is, maximizing light diffusion).

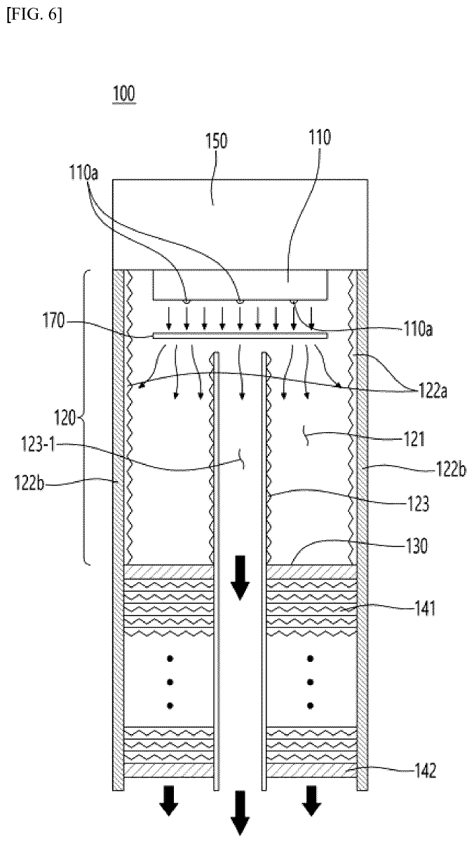

According to one embodiment of the present invention, the ultra-microlight transmission device 100 may include a diffusion plate 170. The diffusion plate 170 will be described in more detail below with reference to FIG. 6.

As shown in FIG. 6, the diffusion plate 170 may form a preset distance from the light source 110 and may diffuse light generated by the light source 110 to transmit the light to the interior space 121 of the housing. According to one embodiment, the diffusion plate 170 may be made of a plastic material such as acryl (polymethyl methacrylate (PMMA)), polycarbonate (PC), or polyethylene terephthalate (PET). Light emitted from the light source 110 is refracted into the diffusion plate 170 and meets a diffusion agent inside the diffusion plate to be repeatedly reflected and dispersed, and in such a process, the light forms constant brightness over the entire surface.

The diffusion plate 170 may refract and diffuse light emitted from the light source 110. As light emitted from the light source 110 is refracted while passing through the diffusion plate 170, the light may be diffused in multiple directions in the interior space 121. Accordingly, light transmitted to the outer wall prism 122a and the inner wall prism 123-2a can be maximized. As the light transmitted to the outer wall prism 122a and the inner wall prism 123-2a is maximized, photoelectronic emission efficiency through the outer wall prism 122a and the inner wall prism 123-2a can be improved. That is, the diffusion plate 170 can maximize a photoelectric effect by diffusing and supplying light to the interior space 121 in which the wall prism is positioned (that is, maximizing light diffusion).

According to one embodiment, the diffusion plate 170 may be provided in a shape with a hollow central portion. In this case, the hollow interior of the diffusion plate 170 may correspond to the hollow interior 123-1 of the second housing 123. Accordingly, the diffusion plate 170 may diffuse and transmit light to be transmitted to a space (for example, the interior space) between the first housing 120 and the second housing 123, and light emitted directly to the outside through the hollow interior may not be diffused or refracted. That is, since the diffusion plate 170 is provided in a shape with a hollow central portion, light may be selectively used. In other words, through the structural features of the diffusion plate 170 as described above, at least a portion of light generated by the light source 110 may be diffused and transmitted to the interior space so as to maximize photoelectric emission efficiency, and the other portion of the light may be emitted directly to the outside without diffusion or refraction.

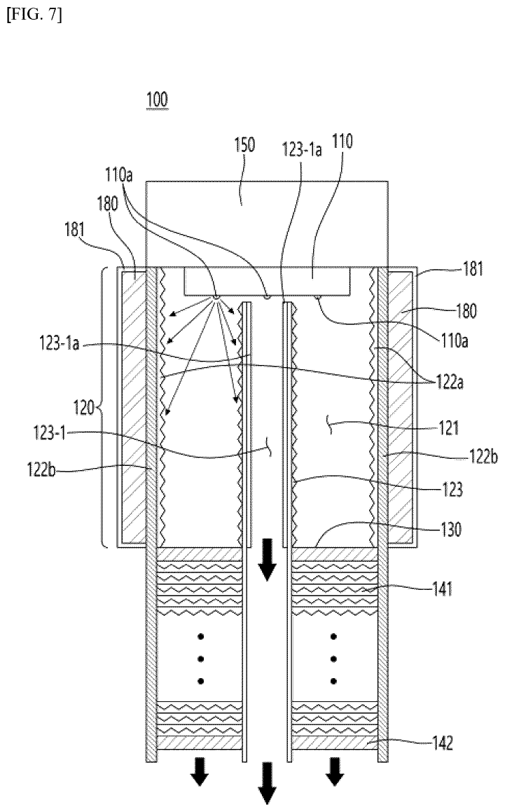

According to one embodiment of the present invention, the ultra-microlight transmission device 100 may include an electromagnetic wave generator 180. As shown in FIG. 7, the electromagnetic wave generator 180 may be provided to surround an outer surface of the first housing 120. The electromagnetic wave generator 180 may be made of a material that generates electromagnetic waves. For example, the electromagnetic wave generator 180 may be made of amphibole that generates electromagnetic waves. In addition, the ultra-microlight transmission device 100 may include a blocking film 181. The blocking film 181 may be used to control one-directional movement of electromagnetic waves. As shown in FIG. 7, the blocking film 181 may be provided to surround an outer surface of the electromagnetic wave generator 180. The blocking film 181 may be for shielding electric fields, magnetic fields, or electromagnetic waves occurring in a direction from inside to outside and made of a metal material such as aluminum (AL) or copper (Cu). When the blocking film 181 is made of a metal material, electromagnetic waves may be reflected from a surface, and an electric field becomes 0 due to the movement of free electrons in a conductor, thereby blocking electromagnetic waves and electric fields in a wide frequency band. The specific description of the materials constituting the above-described blocking film is merely an example, and the present invention is not limited thereto.

That is, the ultra-microlight transmission device 100 may include the electromagnetic wave generator 180 that generates electromagnetic waves between the outer surface of the first housing 120 and the blocking film 181, and a photoelectric effect occurring in the interior space 121 of the first housing 120 can be improved through the electromagnetic waves generated by the electromagnetic wave generator 180. That is, photoelectronic emission efficiency can be maximized by electromagnetic waves. In this case, the blocking film 181 may perform control such that electromagnetic waves generated by the electromagnetic wave generator 180 are not emitted outward. Accordingly, electromagnetic waves are concentrated to the interior space 121, thereby further maximizing a photoelectric effect. In addition, emitted electromagnetic waves are minimized such that an electromagnetic field generated internally do not affect the outside, thereby reducing harmfulness to contribute to an improvement in stability of living creatures.

According to an additional embodiment, the ultra-microlight transmission device 100 may include an internal electromagnetic wave generator 123-1a provided along a circumference of the hollow interior of the second housing 123. Specifically, as shown in FIG. 7, an internal electromagnetic wave generator 123-1a may be formed along the circumference of the hollow interior 123-1 of the second housing 123. The internal electromagnetic wave generator 123-1a may be made of a material that generates electromagnetic waves. For example, the internal electromagnetic wave generator 123-1a may be made of amphibole that generates electromagnetic waves. In this case, since the electromagnetic wave generator 180 and the internal electromagnetic wave generator 123-1a are provided outside the first housing 120 and inside the second housing 123, respectively, photoelectric effect (that is, photoelectronic emission efficiency) can be further maximized in the interior space 121.

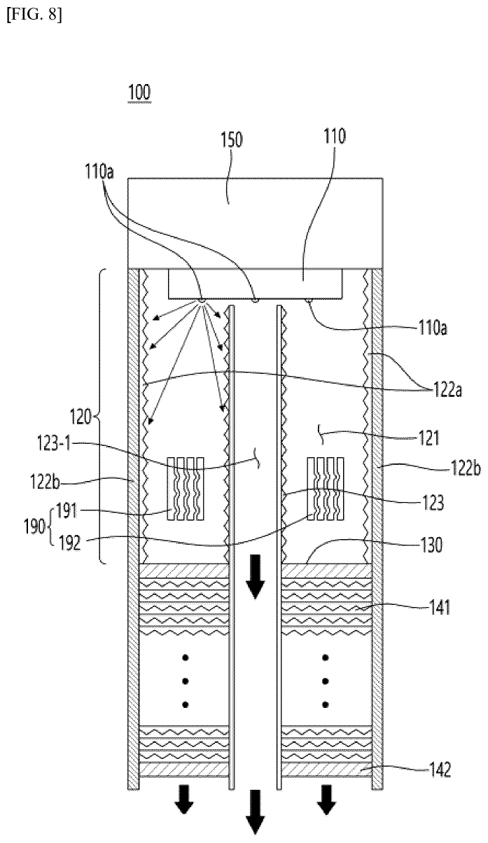

According to one embodiment of the present invention, the ultra-microlight transmission device 100 may include a metal plate 190. The metal plate 190 will be described in more detail below with reference to FIG. 8.

According to one embodiment, as shown in FIG. 8, the metal plate 190 may be provided in one area of the interior space 121 between the first housing 120 and the second housing 123. For example, a plurality of metal plates 190 may be provided between the first housing 120 and the second housing 123. For example, as shown in FIG. 8, a first metal plate 191 and a second metal plate 192 may be provided in the interior space 121 between the first housing 120 and the second housing 123. The description of the number of metal plates described above is merely an example, and the present invention is not limited thereto.

The metal plate 190 may be made of a metal that binds electrons by a positive charge of an atomic nucleus and an electric force. In the metal plate 190, electrons bound within atoms by light may collide with light to be emitted to the outside of metal. That is, additional metal (for example, the metal plate) may be provided in the interior space 121 in addition to the wall prisms (for example, the second outer wall prism and the second inner wall prism), a surface area of the metal capable of emitting photoelectrons is maximized, thereby increasing photoelectronic emission efficiency in the interior space 121. In other words, an area for a photoelectric effect can be improved through the metal plate 190, thereby increasing photoelectronic emission efficiency. According to one embodiment, the metal plate 190 may be made of an aluminum (Al) material. When the metal plate 190 is made of an aluminum material, photoelectronic emission efficiency can be further improved. Specifically, metals have their own unique work function W and limit frequency (or threshold frequency). Here, the work function and the limit frequency may respectively mean the minimum energy and frequency of light that causes electrons bound to metal to be emitted. Aluminum has a work function of 4.06 eV to 4.26 eV, which may be lower than those of other metals. That is, when the metal plate 190 is made of an aluminum material, since the metal plate 190 has a low work function, the minimum energy of light for emitting photoelectrons may be reduced, and thus photoelectrons may be emitted through less light energy. In addition, when the metal plate 190 is made of an aluminum material, thermionic emission efficiency can also be maximized.

In other words, when the metal plate 190 is made of an aluminum material, photoelectronic emission and thermoionic emission efficiencies can be improved. Improvements in photoelectronic emission and thermoionic emission efficiencies may ultimately contribute to an improvement in efficiency of generating ultra-microlight.

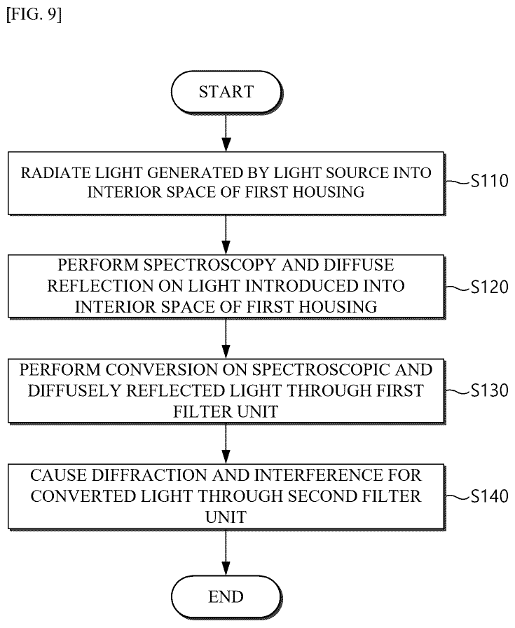

FIG. 9 is a flowchart illustrating a method of generating light energy according to one embodiment of the present invention. According to one embodiment, the method of generating light energy that provides a cell proliferation effect may consist of the following operations. The order of the operations shown in FIG. 9 may be changed as needed, and at least one operation may be omitted or added. That is, the above-described operations are merely one embodiment of the present invention, and the scope of the present invention is not limited thereto.

According to one embodiment of the present invention, the method of generating light energy that provides a cell proliferation effect may include operation S110 of radiating light generated by the light source 110 to the interior space 121 of the first housing 120.

The light source 110 may refer to a radiating body that emits infrared light, visible light, ultraviolet light, or the like. The light source 110 may directly or indirectly convert heat or electrical energy into radiant energy. For example, the light source 110 may generate light related to infrared light, visible light, ultraviolet light, or the like through light-emitting through combustion, light-emitting through electrical discharge, or light-emitting through a semiconductor. The specific description of a light generation method described above is merely an example, and the present invention is not limited thereto. This light source 110 may be disposed in one direction of the first housing 120 to transmit light to the interior space 121 of the first housing 120. In one embodiment, the light source 110 may be constituted through the plurality of LED elements 110a. Each of the plurality of LED elements 110a may be a semiconductor element that generates and provides light through current. Light generated by the light source 110 may be radiated to the interior space 121 of the first housing 120, and as the light strikes a wall in the interior space 121, photoelectrons may be generated.

According to one embodiment of the present invention, the method of generating light energy that provides a cell proliferation effect may include operation S120 of performing spectroscopy and diffuse reflection on light introduced into the interior space 121 of the first housing 120. In this case, the second housing having the shape with the hollow interior may be provided in the interior space 121 of the first housing 120. That is, the ultra-microlight transmission device 100 of the present invention may be constituted through a double housing structure including the second housing 123 inside the first housing 120. In this case, the outer diameter of the second housing 123 may be less than the inner diameter of the first housing 120, and thus the space (that is, the interior space 121) may be formed between the first housing 120 and the second housing 123. In this case, the first housing 120 may be provided to include the outer wall prism 122a in an inward direction, and the second housing 123 may be provided to include the inner wall prism 123-2a in an outward direction. In other words, the wall prisms formed in respective housings may be provided to face each other.

Accordingly, photoelectronic emission and thermionic emission efficiencies can be further maximized. Specifically, since an area of each wall prism (that is, each of the inner wall 122b and the outer wall 123-2b) made of a metal material that binds electrons is further increased, photoelectronic emission and thermal emission can be further maximized. In addition, since light transmitted to each wall prism made of a metal material is light dispersed into photons with a variety of energy through the outer wall prism 122a and the inner wall prism 123-2a, photoelectronic emission can be further maximized.

In summary, the wall prisms may be provided in the first housing 120 and the second housing 123 to face each other, and as a result, the efficiency of spectroscopic and diffusely reflected light can be increases and photoelectron and thermionic emission areas can be increased so that a photoelectric effect and a thermionic emission effect can be maximized.

According to one embodiment of the present invention, the method of generating light energy that provides a cell proliferation effect may include operation S130 of performing conversion on the spectroscopic and diffusely reflected light through the first filter unit 130

The first filter unit 130 may uniformly convert the spectroscopic and diffusely reflected light into monochromatic light to transmit the monochromatic light to the second filter unit 141.

More specifically, the first filter unit 130 may be made of an acrylic material. The first filter unit 130 is provided to have the inner diameter that is greater than the outer diameter of the second housing 123 and may be provided to pass through the second housing 123. For example, the first filter unit 130 may have the outer diameter corresponding to the inner diameter of the first housing 120, may have a thickness of 1 mm to 5 mm, and may pass through the second housing 123. That is, as shown in FIG. 2, the first filter unit 130 may be provided in a donut shape of which an inner diameter corresponds to the outer diameter of the second housing 123.

The first filter unit 130 may be provided to be connected to one end of the first housing 120 and may receive light from the interior space 121 between the first housing 120 and the second housing 123. The light received from the interior space 121 may be light spectroscopic and diffusely reflected through the outer wall prism 122a of the first housing 120 and the inner wall prism 123-2a of the second housing 123 (that is, light through which photoelectronic emission or thermionic emission is performed). Spectroscopic and diffusely reflected light has different white light characteristics according to the intensity and wavelength characteristics of light and thus may exhibit non-uniform color distribution characteristics. Accordingly, the first filter unit 130 may convert the spectroscopic and diffusely reflected light into uniform monochromatic light. For example, the first filter unit 130 may convert the spectroscopic and diffusely reflected light (that is, photoelectrons) into monochromatic light such as blue frequency energy. The first filter unit 130 may function as a color correction filter for light.

That is, light spectroscopic and diffusely reflected from the outer wall prism 122a of the first housing 120 and the inner wall prism 123-2a of the second housing 123 may be converted into uniform monochromatic light while passing through the first filter unit 130 and transmitted to the second filter unit 141 positioned in one direction (for example, downward direction in FIG. 2) of the first filter unit 130. Through the role of the color correction filter of the first filter unit 130, light with various characteristics may be converted into uniform light with the same characteristics.

According to one embodiment of the present invention, the method of generating light energy that provides a cell proliferation effect may include operation S140 of causing diffraction and interference for the light converted through the second filter unit 141.

The second filter unit 141 may be provided to have the inner diameter that is greater than the outer diameter of the second housing 123 and provided to pass through the second housing 123. That is, as shown in FIG. 2, the second filter unit 1430 may be provided in a donut shape of which an inner diameter corresponds to the outer diameter of the second housing 123.

According to one embodiment of the present invention, the ultra-microlight transmission device 100 may include the second filter unit 141 provided by stacking the plurality of prism discs. In addition, the ultra-microlight transmission device 100 may include the third filter unit 142 that filters light transmitted from the second filter unit 141.

In one embodiment, the second filter unit 141 may adjust light converted through continuous diffraction and interference through the plurality of prism discs (that is, light passing through the first filter unit). Specifically, as shown in FIG. 2, the second filter unit 141 may be implemented by stacking the plurality of prism discs.

The second filter unit 141 may be provided in contact with a side of the first filter unit 130 in one direction (for example, a downward direction) and provided in a form in which the plurality of prism discs are stacked. Converted light passing through the first filter unit 130 may be subjected to continuous diffraction and interference while passing through each layer of the second filter unit 141 and thus may be adjusted. Adjusting the converted light may mean that light is adjusted to have an optimal wavelength range, for example, so as to improve cell proliferation efficiency in living creatures. For a specific example, light may be adjusted by passing through the second filter unit 141 and thus may have a wavelength of 300 nm to 870 nm. Here, light with a wavelength of 300 nm to 870 nm may be appropriate light for increasing cell proliferation efficiency (for example, improving reproductive potential) of living creatures. According to one embodiment, the second filter unit 141 may adjust light to have various wavelengths according to an aspect of the provision of the plurality of prism discs. That is, while passing through each layer (that is, the plurality of prism discs), through continuous diffraction and interference, light passing through the second filter unit 141 may be adjusted to have an appropriate wavelength to provide cell proliferation efficiency to living creatures.

In one embodiment, the third filter unit 142 may be made of a black body acrylic material. The black body acrylic material may function as a filter that transmits only light with a specific range of intensity. That is, the third filter unit 142 may allow only light with a certain range of intensity to be emitted to the outside through the black body acrylic material.

Specifically, the third filter unit 142 may emit ultra-microlight to the outside by filtering light with preset intensity among light transmitted from the second filter unit 141. Here, the preset intensity may refer to a range of light related to the optimal intensity for improving cell proliferation efficiency of living creatures. For example, light (that is, ultra-microlight) emitted through the third filter unit 142 may have an intensity of 10⁻¹⁸ W/cm² to 10⁻¹⁵ W/cm². In other words, light with an intensity of 10⁻¹⁸ W/cm² to 10⁻¹⁵ W/cm² may be light with optimal intensity for increasing cell proliferation efficiency of living creatures. For example, when light outside a range of 10⁻¹⁸ W/cm² to 10⁻¹⁵ W/cm² (for example, light with an intensity of 10⁻¹¹ W/cm²) is radiated onto living creatures, the light may not be appropriate light (that is, ultra-microlight) that increases cell proliferation efficiency of living creatures.

That is, the third filter unit 142 may filter light (for example, light in a specific wavelength band) passing through the second filter unit 141 such that only light with specific intensity is emitted to the outside. Accordingly, the light emitted to the outside may be ultra-microlight which is light with optimal intensity for increasing cell proliferation efficiency of living creatures.

Meanwhile, it can be confirmed through the following experimental process and results that an antibody production function of mammals administered a vaccine is improved through ultra-microlight according to one embodiment of the present invention. Through the following experiments, the effects of ultra-microlight on the growth performance, immune system, and metabolism of mammals could be confirmed. The experiment was performed through a PED-X® vaccine of CAVC. PED-X® is a vaccine against porcine epidemic diarrhea (PED) virus type 2b which is currently circulating outdoors. PED-X® may amplify an immunoglobulin A (IgA) antibody and sustain the formation thereof. The vaccine used in the experiment was PED-X®, but similar results could be obtained in experiments using other vaccines (for example, SuiShot CSFV Marker-L, SuiShot CSFM-B, APM-X, and AR-X).

In an embodiment, an experiment was performed on an experimental group (that is, an experimental group radiated with ultra-microlight) and a control group which were formed through a total of 30 pigs that had an average initial body weight (BW) of 7.06±0.11 kg and were 21 days old.

The experiment was performed in a metal cage with a plastic floor (1.2 m×2.4 m), an average temperature of the cage was maintained in a range of 25° C. to 30° C., and humidity was maintained in a range of 61% to 66%.

The experiment was performed for 48 days, and after vaccine administration, values measured from each of the experimental and control groups were recorded on each of days 14, 24, and 48. Here, the experimental group refers to pigs that were radiated with ultra-microlight of the present invention for at least 2 hours a day.

In this case, the intensity of the ultra-microlight was too weak to be measured using a spectrometer, and thus an intensity value was measured as a value measured 2 cm in front of an end surface of a light radiation device. Meanwhile, since the intensity of light is attenuated in inverse proportion to (distance)², when installed in an actual pig pen, the light radiation device is installed at a circle having a radius of about 2 m to 5 m from a mammal. It was confirmed that the final intensity of the light source was in a range of 10⁻¹⁸ W/cm² to 10⁻¹⁵ W/cm².

TABLE 1
Growth performance

| Item | Control group | Experimental group | P-value |
|---|---|---|---|
| Initial BW (kg) | 7.07 | 7.05 | 0.992 |
| Final BW (kg) | 31.7 | 34.17 | 0.08 |
| d 14 | | | |
| ADG (g) | 371 | 395 | 0.204 |
| ADFI (g) | 512 | 522 | 0.447 |
| G:F | 0.725 | 0.756 | 0.27 |
| d 28 | | | |
| ADG (g) | 455 | 521 | 0.065 |
| ADFI (g) | 679 | 736 | 0.121 |
| G:I | 0.67^{b} | 0.71ª | 0.039 |
| d 48 | | | |
| ADG (g) | 653 | 715 | 0.178 |
| ADFI (g) | 1,145 | 1,181 | 0.622 |
| G:F | 0.57^{b} | 0.61ª | 0.018 |
| Overall | | | |
| ADG (g) | 513 | 565 | 0.088 |
| ADFI (g) | 825 | 859 | 0.28 |
| G:F | 0.62 | 0.66 | 0.113 |

Looking at [Table 1], it could be confirmed that the experimental group irradiated with ultra-microlight gained more weight than the initial BW as compared to the control group that was not radiated with ultra-microlight. Specifically, in the experimental group (that was radiated with ultra-microlight), an average initial BW of 15 pigs was 7.07 kg, but after 48 days, the weight was 34.17 kg, which was increased by 27.1 kg. In the control group (that was not radiated with ultra-microlight), an average initial BW of 15 pigs was 7.07 kg, but it could be confirmed that, after 48 days, the BW was 31.7 kg, which was increased by 24.63 kg. That is, it could be confirmed that the experimental group radiated with ultra-microlight gained 2.47 kg as compared to the control group radiated with ultra-microlight. In particular, it could be confirmed that measured values of average daily feed intake (ADFI) and average daily gain (ADG) in the experimental group were higher than those in the control group on all days 14, 24, and 48.

In addition, in the case of the experimental group, it can be seen that a gain to feed ratio (G:F) is consistently higher than that of the control group, and it can be confirmed that a P-value (reliability value of corresponding information) thereof is 0.05 or less, which is very reliable information.

That is, as in the above-described experimental results, in the case of the pigs radiated with ultra-microlight, the ADFI, the ADG, and the G:F considerably increased as compared to the pigs not radiated with light, and it could be confirmed that the total BI gain considerably increased. In other words, it can be confirmed that when ultra-microlight with specific intensity and wavelength is radiated for at least 2 hours a day, the growth performance of mammals is improved.

In addition, blood samples were collected using disposable vacuum tubes without an anticoagulant manufactured by Becton, Dickinson and Company to correspond to the experimental and control groups. Serum samples were centrifuged for 15 minutes and stored at a temperature of −20° C., and then analysis was performed on each sample. A hematology system (Drew Scientific Co. LTD, Oxford, CT) was performed and an ELISA kit was used to obtain measured values of immunoglobulin G (IgG), IgA, IL-1β, TNF-α, and IL-6.

TABLE 2
Blood analysis

| Item | Control group | Experimental group | P-value |
|---|---|---|---|
| d 28 | | | |
| IgA (ng/ml) | 8.72^{b} | 10.28ª | 0.001 |
| IgG (ng/ml) | 22.59^{b} | 26.12ª | 0.001 |
| d 48 | | | |
| IgA (ng/ml) | 33.76^{b} | 58.41ª | <0.001 |
| IgG (ng/ml) | 41.56^{b} | 45.15ª | 0.036 |

Immunoglobulin is a glycoprotein molecule produced in an immune response upon stimulation by an antigen and mainly binds specifically to a specific antigen in the blood to cause an antigen-antibody reaction. In an embodiment, the immunoglobulin is referred to as an antibody and produced from B lymphocytes to perform a function of removing antigens from pathogenic microorganisms such as bacteria and viruses through precipitation or an agglutination reaction. In addition, the immunoglobulin induces various immune functions through interactions with other elements of an immune system. In other words, a higher level of the immunoglobulin may mean that an immune function is improved. Referring to [Table 2], it could be confirmed that both IgA and IgG showed high levels in the experimental group radiated with ultra-microlight. That is, it could be confirmed that an immune function of the experimental group radiated with ultra-microlight for at least 2 hours a day for 48 days was considerably improved. In particular, it can be confirmed that the P-value (reliability value of the information) corresponding to each group is than 0.05 or less, which is very reliable information

In the above, although embodiments of the present invention have been described with reference to the accompanying drawings, those skilled in the art may understand that the present invention may be embodied in other specific forms without changing the technical spirit or essential features thereof. Accordingly, it should be understood that the above-described embodiments are exemplary in all respects and not restrictive.

(Description of Reference Numerals)

| | |
|---|---|
| 11: indoor space | |
| 100: ultra-microlight transmission device | 110: light source |
| 110a: plurality of LED elements | 120: first housing |
| 121: interior space | 122a: outer wall prism |
| 122b: inner wall | 123: second housing |
| 123-1: hollow interior | 123-1a: internal electromagnetic wave generator |
| 123-2a: inner wall prism | 123-2b: outer wall |
| 130: first filter unit | 141: second filter unit |
| 142: third filter unit | 150: heat radiation member |
| 160: one or more lenses | 170: diffusion plate |
| 180: electromagnetic wave generator | 181: blocking film |
| 190: metal plate | 191: first metal plate |
| 192: second metal plate | |

### MODES OF THE INVENTION

The best mode for carrying out the present invention has been described above.

### INDUSTRIAL APPLICABILITY

The present invention can be utilized in the field of activating the biometabolism of living creatures and strengthen immunity.

1. An ultra-microlight transmission device having a double housing structure, comprising:
a light source configured to generate light;
a first housing which includes an interior space and performs spectroscopy and diffuse reflection on light introduced into the interior space;
a second housing which has a shape with a hollow interior and is provided in the interior space of the first housing;
a first filter unit configured to convert the spectroscopic and diffusely reflected light into monochromatic light; and
a second filter unit configured to cause diffraction and interference for the converted light.

2. The ultra-microlight transmission device having a double housing structure of claim 1, wherein at least a portion of the light generated by the light source is directly emitted to an outside through the hollow interior of the second housing.

3. The ultra-microlight transmission device having a double housing structure of claim 1, further comprising:
a heat radiation member configured to absorb heat generated by the light source and transfer the absorbed heat to the interior space.

4. The ultra-microlight transmission device having a double housing structure of claim 1, wherein the first housing includes an outer wall prism provided therein and configured to perform spectroscopy and diffuse reflection on the introduced light in multiple directions,
the second housing includes an inner wall prism provided at an outer circumference thereof and configured to perform spectroscopy and diffuse reflection on the introduced light in multiple directions, and
the light spectroscopic and diffusely reflected by the outer wall prism and the inner wall prism is radiated to the housing to emit photoelectrons to the interior space.

5. The ultra-microlight transmission device having a double housing structure of claim 4, wherein an inner wall of the first housing is made of a stainless steel material, and
the outer wall prism is made of an acrylic material and supported on the inner wall.

6. The ultra-microlight transmission device having a double housing structure of claim 1, wherein the second housing further includes an internal electromagnetic wave generator provided in the hollow interior.

7. The ultra-microlight transmission device having a double housing structure of claim 1, wherein each of the first filter unit and the second filter unit is provided to have an inner diameter that is greater than an outer diameter of the second housing and provided to pass through the second housing.

8. The ultra-microlight transmission device having a double housing structure of claim 1, wherein the second filter unit adjusts the converted light by causing continuous diffraction and interference through a plurality of prism discs.

9. The ultra-microlight transmission device having a double housing structure of claim 1, further comprising:
a third filter unit configured to perform filtering on light transmitted from the second filter unit,
wherein the third filter unit is made of a black body acrylic plate material and filters light with predetermined energy intensity among the light transmitted from the second filter unit to emit the filtered light to an outside.

10. The ultra-microlight transmission device having a double housing structure of claim 1, further comprising:
one or more lenses configured to refract the light generated by the light source and transmit the refracted light to the interior space.

11. The ultra-microlight transmission device having a double housing structure of claim 1, further comprising:
a diffusion plate which is provided at a predetermined distance from the light source and diffuses the light generated by the light source to transmit the diffused light to the interior space.

12. The ultra-microlight transmission device having a double housing structure of claim 1, further comprising:
an electromagnetic wave generator provided to surround an outer surface of the first housing and configured to generate electromagnetic waves; and
a blocking film provided to surround an outer surface of the electromagnetic wave generator and configured to block one-directional movement of the electromagnetic waves.

13. The ultra-microlight transmission device having a double housing structure of claim 1, further comprising:
a metal plate provided in one area of the interior space.

14. A method of generating light energy, the method comprising:
radiating light generated by a light source to an interior space of a first housing;
performing spectroscopy and diffuse reflection on the light introduced into the interior space of the first housing;
performing conversion on the spectroscopic and diffusely reflected light through a first filter unit; and
causing diffraction and interference for the converted light through a second filter unit,
wherein a second housing having a hollow interior is provided in the interior space of the first housing.
